(19) United States
(12) Patent Application Publication
(10) Pub. No.: US 2024/0265543 A1
(43) Pub. Date: Aug. 8, 2024

(54) MEDICAL IMAGE PROCESSING APPARATUS, METHOD, AND STORAGE MEDIUM

(71) Applicant: CANON MEDICAL SYSTEMS CORPORATION, Tochigi (JP)

(72) Inventor: Takuya SAKAGUCHI, Utsunomiya (JP)

(73) Assignee: CANON MEDICAL SYSTEMS CORPORATION, Tochigi (JP)

(21) Appl. No.: 18/430,114

(22) Filed: Feb. 1, 2024

(30) Foreign Application Priority Data
Feb. 2, 2023 (JP) 2023-014769

(51) Int. Cl. G06T 7/00; G06T 7/33

(52) U.S. Cl. CPC G06T 7/0014; G06T 7/337; G06T 2207/10081; G06T 2207/10116; G06T 2207/30104

(57) ABSTRACT

A medical image processing apparatus according to an embodiment includes processing circuitry. The processing circuitry is configured to obtain a graph of an index value related to a blood flow based on a CT image and a graph of an index value related to the blood flow based on an angiography image. On the basis of shapes of the graphs of the index values related to the blood flow, the processing circuitry is configured to determine a target position for performing a position alignment between the graph of the index value related to the blood flow based on the CT image and the graph of the index value related to the blood flow based on the angiography image. On the basis of the target position, the processing circuitry is configured to perform the position alignment between the graph of the index value related to the blood flow based on the CT image and the graph of the index value related to the blood flow based on the angiography image.

### CROSS-REFERENCE TO RELATED APPLICATIONS

This application is based upon and claims the benefit of priority from Japanese Patent Application No. 2023-014769, filed on Feb. 2, 2023; the entire contents of which are incorporated herein by reference.

### FIELD

Embodiments described herein relate generally to a medical image processing apparatus, a method, and a storage medium.

### BACKGROUND

A technique called “Fractional Flow Reserve (FFR)” is known and is used for measuring blood pressure in a coronary artery of the heart. Around the year 2015, a method for putting a pressure sensor in a blood vessel was brought into practical use. Around the year 2020, a method for estimating pressure by using an image was brought into general use. In recent years, there have been many reports indicating that a method by which an FFR is measured from an angiography image (hereinafter, “Angio-FFR”) is able to achieve a result comparable to that achieved by the method using a pressure sensor (hereinafter, “wire FFR”). If this development continues on, there is a high possibility that Angio-FFR may replace the wire FFR technique. The Angio-FFR technique requires only angiography imaging and thus has an advantage over the wire FFR technique because it is possible to safely acquire the value, without the need to make wires cross in all of the three blood vessel branches at once to reach the peripheral end of a stenosis.

### BRIEF DESCRIPTION OF THE DRAWINGS

FIG. 1 is a drawing for explaining an example of a workflow according to a first embodiment;

FIG. 2 is a diagram illustrating an exemplary configuration of a medical image processing apparatus according to the first embodiment;

FIG. 3 is a flowchart illustrating a processing procedure of processes performed by processing functions included in processing circuitry of the medical image processing apparatus according to the first embodiment;

FIG. 4 is a drawing for explaining an example of a position alignment process according to the first embodiment;

FIG. 5 is a drawing illustrating an example of a comparison display according to the first embodiment;

FIG. 6A is a drawing illustrating an example of display control exercised by a controlling function according to the first embodiment;

FIG. 6B is a drawing illustrating another example of the display control exercised by the controlling function according to the first embodiment;

FIG. 6C is a drawing illustrating yet another example of the display control exercised by the controlling function according to the first embodiment;

FIG. 6D is a drawing illustrating yet another example of the display control exercised by the controlling function according to the first embodiment;

FIG. 7 is a drawing illustrating another example of the comparison display according to the first embodiment;

FIG. 8 is a diagram illustrating an exemplary configuration of a medical image processing apparatus according to a second embodiment;

FIG. 9 is a flowchart illustrating a processing procedure of processes performed by processing functions included in processing circuitry of the medical image processing apparatus according to the second embodiment;

FIG. 10 is a drawing for explaining an example of an image display according to the second embodiment; and

FIG. 11 is a drawing illustrating an example of display control according to the second embodiment.

### DETAILED DESCRIPTION

A medical image processing apparatus according to an embodiment includes processing circuitry. The processing circuitry is configured to obtain a graph of an index value related to a blood flow based on a Computed Tomography (CT) image and a graph of an index value related to the blood flow based on an angiography image. On the basis of shapes of the graphs of the index values related to the blood flow, the processing circuitry is configured to determine a target position for performing a position alignment between the graph of the index value related to the blood flow based on the CT image and the graph of the index value related to the blood flow based on the angiography image. On the basis of the target position, the processing circuitry is configured to perform the position alignment between the graph of the index value related to the blood flow based on the CT image and the graph of the index value related to the blood flow based on the angiography image.

Exemplary embodiments of a medical image processing apparatus, a method, and a program will be explained in detail below, with reference to the accompanying drawings. The medical image processing apparatus, the method, and the program of the present disclosure are not limited to the embodiments described below. Further, in the explanations below, some of the constituent elements that are the same as each other will be referred to by using the same reference characters, and duplicate explanations thereof will be omitted. Also, in the present embodiments, FFR will be used as an example of the index value related to the blood flow.

### FIRST EMBODIMENT

As explained above, Angio-FFR has been gaining attention in recent years. Presently, as a pre-surgery procedure, a method (hereinafter, “CT-FFR”) by which an FFR value is measured from a CT image is widely used. If both CT-FFR and Angio-FFR start being widely used, because both of the techniques visualize mutually the same phenomenon related to a contrast agent flow of an iodine contrast agent while using mutually the same type of image based on an X-ray absorption principle, making a comparison on the medical physiological phenomenon will be easy. In addition, the fact that both of the techniques are free from shape changes caused by insertion of a wire also makes the comparison easy. As explained herein, CT-FFR and Angio-FFR have excellent compatibility with each other in making the comparison. However, because these techniques are based on the images acquired at mutually-different times under mutually-different conditions, simply making the comparison will result in a low level of precision. To cope with this situation, the present embodiments will propose technical methods that make it possible to easily compare, match, and observe the two with an excellent level of precision, by applying improvements to position alignment and display methods.

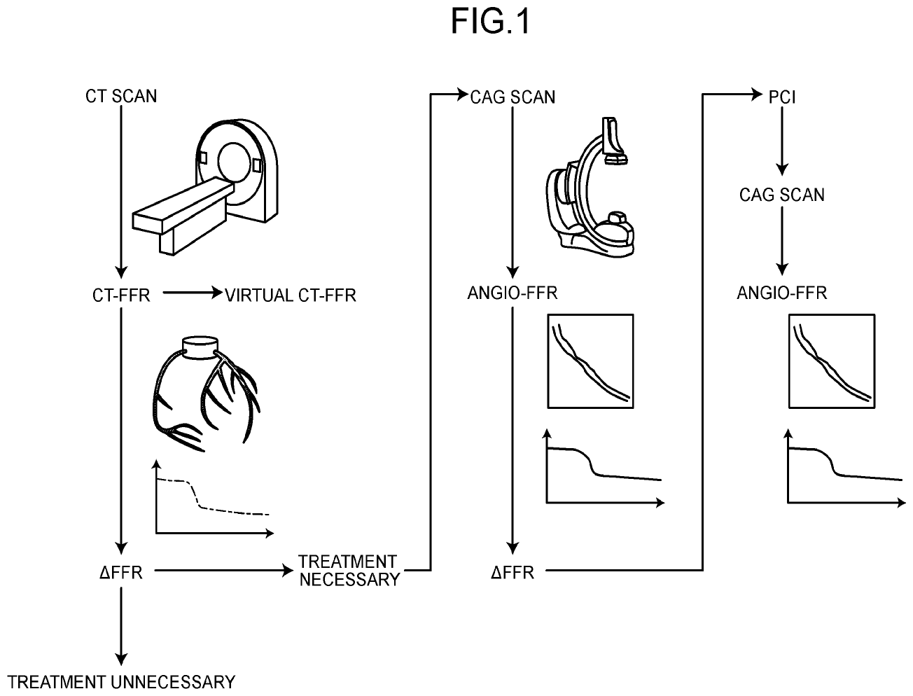

To begin with, a workflow of a manipulation to which the present embodiments are applied will be explained. FIG. 1 is a drawing for explaining an example of a workflow according to a first embodiment. As illustrated in FIG. 1, during the manipulation to which a medical image processing apparatus, a method, and a program of the present embodiment are applied, a CT image is acquired by performing a CT scan, and an FFR value based on the CT image is estimated (“CT-FFR” in the drawing). In this situation, if treatment is required subsequently, a simulation is performed with virtual treatment so as to estimate how the FFR value will transition after the treatment (“Virtual CT-FFR” in the drawing).

When FFR values based on the CT image have been estimated, a difference (ΔFFR) between FFR values is calculated with respect to each of various positions in the blood vessel, so as to determine whether or not the treatment is required. In this situation, when there is a location exhibiting a large ΔFFR value (i.e., a location where the blood flow is obstructed and a large pressure gradient is exhibited), the treatment will be performed while using the location as a treated site. On the contrary, when the ΔFFR values are small (i.e., there is no location exhibiting a drastic pressure gradient), the blood flow is considered to have no abnormalities, and no treatment will be performed.

When the treatment is determined to be necessary, the patient is subsequently sent to a treatment room. In the treatment room, a Coronary Angiography (CAG) scan is performed by using an angiography apparatus. In this situation, Angio-FFR values are measured before the surgery (pre-surgery) and after the surgery (post-surgery). For example, after the patient entered the treatment room, the CAG scan is performed by the angiography apparatus before the treatment is performed, so as to estimate an FFR value based on the angiography image (“Angio-FFR” in the drawing). In other words, before the treatment is actually started, the location exhibiting the large ΔFFR value (the location where the blood flow is obstructed and the large pressure gradient is exhibited) is checked, so as to confirm that the location is the same as the location specified by CT-FFR.

In this situation, when it is confirmed that the result of Angio-FFR is the same as the result of CT-FFR, insertion of a treatment tool is prepared. In contrast, when the result of Angio-FFR is different from the result of CT-FFR, the cause will be searched for, and the insertion of the treatment tool will not be carried out until the problem is solved. The medical image processing apparatus, the method, and the program of the present embodiment will be applied to this situation and make it possible to appropriately compare the result of CT-FFR with the result of Angio-FFR.

Further, when it is determined that the treatment tool will be inserted, a medical tool (e.g., a stent or a balloon) will be inserted to implement a Percutaneous Coronary Intervention (PCI), so as to perform treatment to resolve the blood flow obstructed location. Subsequently, after the medical tool is inserted, it is checked at that moment whether or not the blood flow obstructed location has been resolved. In other words, a CAG scan is performed in the same state as before the medical tool was inserted, and an Angio-FFR value is estimated to confirm that the ΔFFR value became smaller. The medical image processing apparatus, the method, and the program according to the present embodiment are also applied to this situation and make it possible to appropriately compare the result of CT-FFR with the result of Angio-FFR.

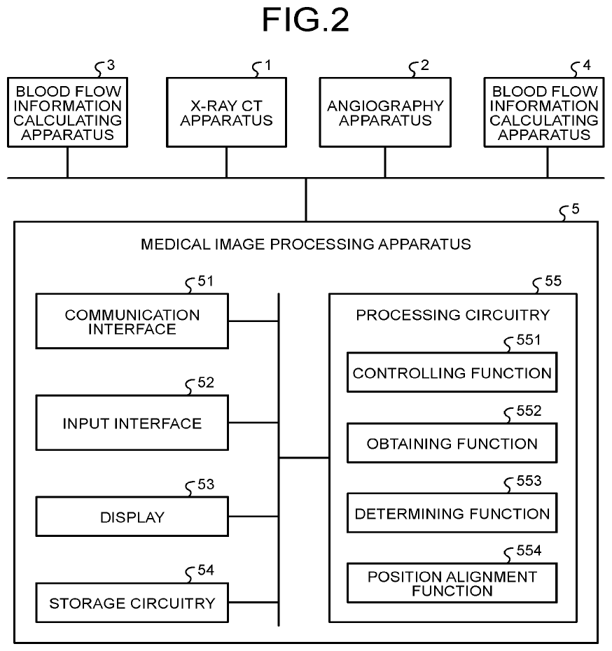

Next, the medical image processing apparatus according to the present embodiment will be explained. FIG. 2 is a diagram illustrating an exemplary configuration of a medical image processing apparatus 5 according to the first embodiment. For example, as illustrated in FIG. 2, the medical image processing apparatus 5 according to the present embodiment is connected to an X-ray Computed Tomography (CT) apparatus 1, an angiography apparatus 2, a blood flow information calculating apparatus 3, and a blood flow information calculating apparatus 4, so as to be able to communicate via a network. To the network illustrated in FIG. 2, other various types of apparatuses (e.g., an image storing apparatus) and systems may also be connected.

The X-ray CT apparatus 1 is configured to acquire a CT image (volume data) of an examined subject (hereinafter, “patient”). More specifically, the X-ray CT apparatus 1 is configured to cause an X-ray tube and an X-ray detector to make a rotating movement while being substantially centered on the heart of the patient to whom a contrast agent was administered and is configured to acquire projection data by detecting X-rays that have passed through the patient. After that, on the basis of the acquired projection data, the X-ray CT apparatus 1 is configured to generate the CT image. In an example, the X-ray CT apparatus 1 is configured to generate a contrast-enhanced coronary artery CT image used for calculating an index value related to the blood flow in a coronary artery.

The angiography apparatus 2 is configured to acquire a two-dimensional projection data (an angiography image) of the patient. More specifically, the angiography apparatus 2 is configured to position an arm holding an X-ray tube on one end thereof and holding an X-ray detector on the other end thereof, so that X-rays are emitted from a prescribed direction onto the heart of the patient to whom the contrast agent was administered, and is configured to acquire two-dimensional projection data by detecting X-rays that have passed through the patient. In an example, the angiography apparatus 2 is configured to acquire two-dimensional angiography projection data used for calculating an index value related to the blood flow in the coronary artery. In this situation, when being configured as a biplane apparatus, the angiography apparatus 2 is also capable of simultaneously acquiring two-dimensional angiography projection data from multiple directions.

The blood flow information calculating apparatus 3 is configured to calculate the index value related to the blood flow by using the contrast-enhanced coronary artery CT image generated by the X-ray CT apparatus 1. For example, the blood flow information calculating apparatus 3 is configured to calculate the index value related to the blood flow in the coronary artery of the patient, by generating a three-dimensional model of the coronary artery while using the contrast-enhanced coronary artery CT image corresponding to a single temporal phase or multiple temporal phases and further performing an analysis on the generated three-dimensional model. Alternatively, the blood flow information calculating apparatus 3 may calculate the index value by performing an analysis on the contrast-enhanced coronary artery CT image without generating the three-dimensional model. Further, examples of the abovementioned analysis include a fluid analysis using Navier-Stokes equations and machine learning. However, possible embodiments are not limited to these examples. It is acceptable to use any applicable method.

The blood flow information calculating apparatus 4 is configured to calculate the index value related to the blood flow, by using the two-dimensional angiography projection data acquired by the angiography apparatus 2. For example, the blood flow information calculating apparatus 4 is configured to calculate the index value related to the blood flow in the coronary artery of the patient, by generating a three-dimensional model of the coronary artery while using the two-dimensional angiography projection data and further performing an analysis on the generated three-dimensional model. Further, examples of the abovementioned analysis include a fluid analysis using Navier-Stokes equations and machine learning. However, possible embodiments are not limited to these examples. It is acceptable to use any applicable method.

The medical image processing apparatus 5 is configured to perform various types of information processing processes related to the patient. More specifically, the medical image processing apparatus 5 is configured to receive, via the network, the index values (the FFR values) related to the blood flow, from the blood flow information calculating apparatus 3 and from the blood flow information calculating apparatus 4 and is configured to perform the various types of information processing processes by using the index values. For example, the medical image processing apparatus 5 is realized by using a computer machine such as a server or a workstation.

For example, the medical image processing apparatus 5 includes a communication interface 51, an input interface 52, a display 53, storage circuitry 54, and processing circuitry 55.

The communication interface 51 is configured to control transfer of various types of data and communication performed between the medical image processing apparatus 5 and the other apparatuses connected via the network. More specifically, the communication interface 51 is connected to the processing circuitry 55 and configured to transmit data received from the other apparatuses to the processing circuitry 55 and to transmit data transmitted thereto from the processing circuitry 55 to the other apparatuses. For example, the communication interface 51 is realized by using a network card, a network adaptor, a Network Interface Controller (NIC), or the like.

The input interface 52 is configured to receive, from a user, operations to input various types of instructions and various types of information. More specifically, the input interface 52 is connected to the processing circuitry 55 and configured to convert the input operations received from the user into electrical signals and to transmit the electrical signals to the processing circuitry 55. For example, the input interface 52 is realized by using a trackball, a switch button, a mouse, a keyboard, a touchpad on which input operations can be performed by touching an operation surface thereof, a touch screen in which a display screen and a touchpad are integrally formed, a contactless input interface using an optical sensor, an audio input interface, and/or the like. In the present disclosure, the input interface 52 does not necessarily need to include physical operation component parts such as the mouse, the keyboard, and/or the like. For instance, possible examples of the input interface 52 include electrical signal processing circuitry configured to receive an electrical signal corresponding to an input operation from an external input mechanism provided separately from the apparatus and to transmit the electrical signal to controlling circuitry.

The display 53 is configured to display various types of information and various types of data. More specifically, the display 53 is connected to the processing circuitry 55 and configured to display the various types of information and the various types of data received from the processing circuitry 55. For example, the display 53 is realized by using a liquid crystal display, a Cathode Ray Tube (CRT) display, a touch panel, or the like.

The storage circuitry 54 is configured to store therein various types of data and various types of programs. More specifically, the storage circuitry 54 is connected to the processing circuitry 55 and configured to store therein data received from the processing circuitry 55 and to read and transmit any of the data stored therein to the processing circuitry 55. For example, the storage circuitry 54 is realized by using a semiconductor memory element such as a Random Access Memory (RAM) or a flash memory, or a hard disk, an optical disk, or the like.

The processing circuitry 55 is configured to control the entirety of the medical image processing apparatus 5. For example, the processing circuitry 55 is configured to perform various types of processes in accordance with the input operations received from the user via the input interface 52. For example, the processing circuitry 55 is configured to receive, via the communication interface 51, data transmitted from another apparatus and to further store the received data into the storage circuitry 54. Also, by transmitting any of the data received from the storage circuitry 54 to the communication interface 51, the processing circuitry 55 is configured to transmit the data to any of the other apparatuses. In addition, for example, the processing circuitry 55 is configured to display any of the data received from the storage circuitry 54 on the display 53.

An exemplary configuration of the medical image processing apparatus 5 of the present embodiment has thus been explained. For example, the medical image processing apparatus 5 according to the present embodiment is installed in a medical facility such as a hospital or a clinic (e.g., in a treatment room where the angiography apparatus 2 is installed) and is configured to assist various types of diagnosing processes, treatment plan creation, and the like performed by the user such as a medical doctor. For example, the medical image processing apparatus 5 is configured to perform various types of processes for appropriately making a comparison between the index value related to the blood flow based on the CT image and the index value related to the blood flow based on the angiography image. Next, details of the medical image processing apparatus 5 will be explained.

For example, as illustrated in FIG. 2, in the present embodiment, the processing circuitry 55 of the medical image processing apparatus 5 executes a controlling function 551, an obtaining function 552, a determining function 553, and a position alignment function 554. In this situation, the controlling function 551 is an example of a display controlling unit. The obtaining function 552 is an example of an obtaining unit. The determining function 553 is an example of a determining unit. The position alignment function 554 is an example of a position alignment unit.

In accordance with operations performed via the input interface 52, the controlling function 551 is configured to exercise control so that various types of GUIs (Graphical User Interfaces) and various types of display information are generated and displayed on the display 53. For example, the controlling function 551 is configured to cause the display 53 to display the index value related to the blood flow based on the CT image and the index value related to the blood flow based on the angiography image. Further, the controlling function 551 is configured to generate various types of display images on the basis of the CT image and the angiography image used for calculating the index values related to the blood flow and to cause the display 53 to display the generated display images.

For example, the controlling function 551 is configured to display, in a superimposed manner, a graph of index values related to the blood flow based on the CT image and a graph of index values related to the blood flow based on the angiography image on which a position alignment has been performed by the position alignment function 554. Processes performed by the controlling function 551 will be explained in detail later.

The obtaining function 552 is configured to obtain, via the communication interface 51, various types of information from various types of apparatuses connected to the network. For example, the obtaining function 552 is configured to obtain the graph of the index values related to the blood flow based on the CT image and the graph of the index values related to the blood flow based on the angiography image. In other words, the obtaining function 552 is configured to obtain an index value (a CT-FFR value) related to the blood flow in each of various positions (various positions along the extending direction of the blood vessel) in the blood vessel rendered in the CT image and an index value (an Angio-FFR value) related to the blood flow in each of the various positions in the blood vessel rendered in the angiography image. Further, the obtaining function 552 is also capable of obtaining the CT image used for calculating the CT-FFR values and the angiography image used for calculating the Angio-FFR values from the X-ray CT apparatus 1, the angiography apparatus 2, the blood flow information calculating apparatus 3, and/or the blood flow information calculating apparatus 4. Processes performed by the obtaining function 552 will be explained in detail later.

On the basis of the shapes of the graphs of the index values related to the blood flow, the determining function 553 is configured to determine a target position for performing a position alignment between the graph of the index values (the CT-FFR values) related to the blood flow based on the CT image and the graph of the index values (the Angio-FFR values) related to the blood flow based on the angiography image. More specifically, the determining function 553 is configured to determine the target position on the basis of the difference between the index values related to the blood flow with respect to each of the various positions in the blood vessel rendered in the CT image. Processes performed by the determining function 553 will be explained in detail later.

On the basis of the target position, the position alignment function 554 is configured to perform the position alignment between the graph of the index values (the CT-FFR values) related to the blood flow based on the CT image and the graph of the index values (the Angio-FFR values) related to the blood flow based on the angiography image. More specifically, the position alignment function 554 is configured to perform the position alignment between the graph indicating the CT-FFR values in the various positions along the extending direction of the blood vessel and the graph indicating the Angio-FFR values in the various positions along the extending direction of the blood vessel. Processes performed by the position alignment function 554 will be explained in detail later.

The processing circuitry 55 described above is realized by using a processor, for example. In that situation, the abovementioned processing functions are stored in the storage circuitry 54 in the form of computer-executable programs. Further, the processing circuitry 55 is configured to realize the functions corresponding to the programs, by reading and executing the programs stored in the storage circuitry 54. In other words, the processing circuitry 55 that has read the programs has the processing functions illustrated in FIG. 2.

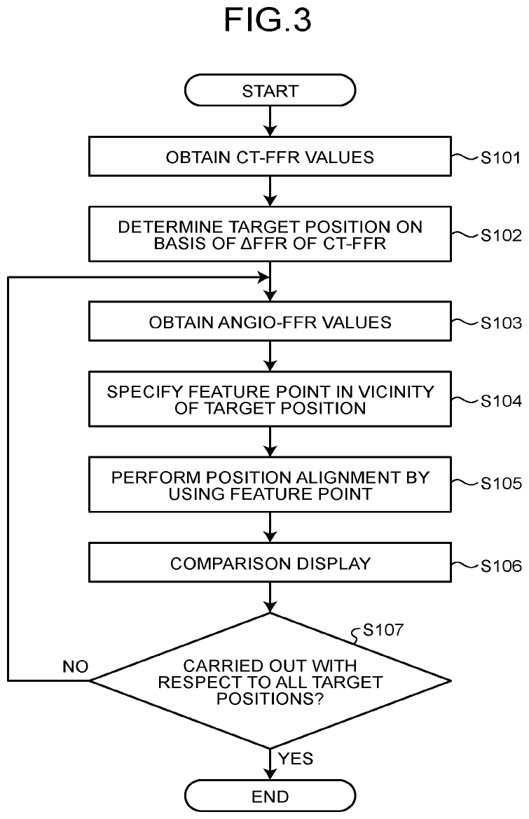

Next, a procedure of processes performed by the medical image processing apparatus 5 will be explained with reference to FIG. 3, and details of each of the processes will subsequently be explained. FIG. 3 is a flowchart illustrating the processing procedure of the processes performed by the processing functions included in the processing circuitry 55 of the medical image processing apparatus 5 according to the first embodiment. The flowchart illustrated in FIG. 3 is applicable to both a pre-surgery CAG scan performed after the patient determined to undergo treatment has entered a treatment room and a post-surgery CAG scan.

For example, as illustrated in FIG. 3, in the present embodiment, the obtaining function 552 obtains the CT-FFR values of the patient from the blood flow information calculating apparatus 3 (step S101). For example, in response to a CT-FFR value obtaining operation performed via the input interface 52, the obtaining function 552 obtains the CT-FFR values that were obtained pre-surgery. This process is realized, for example, as a result of the processing circuitry 55 invoking and executing the program corresponding to the obtaining function 552 from the storage circuitry 54.

Subsequently, on the basis of the ΔFFR values of the obtained CT-FFR values, the determining function 553 is configured to determine a target position (step S102). This process is realized, for example, as a result of the processing circuitry 55 invoking and executing the program corresponding to the determining function 553 from the storage circuitry 54.

After that, the obtaining function 552 obtains the angio-FFR values of the patient from the blood flow information calculating apparatus 4 (step S103). This process is performed after the CAG scan is performed on the patient who entered the treatment room and the blood flow information calculating apparatus 4 has calculated the angio-FFR values. Further, this process is realized, for example, as a result of the processing circuitry 55 invoking and executing the program corresponding to the obtaining function 552 from the storage circuitry 54.

Subsequently, the position alignment function 554 specifies a feature point in the vicinity of the target position (step S104) and performs the position alignment by using the feature point (step S105). This process is realized, for example, as a result of the processing circuitry 55 invoking and executing the program corresponding to the position alignment function 554 from the storage circuitry 54.

After that, on the basis of the result of the position alignment, the controlling function 551 carries out a comparison display between the graph indicating the CT-FFR values and the graph indicating the Angio-FFR values (step S106). More specifically, on the basis of the result of the position alignment, the controlling function 551 causes the display 53 provided in the treatment room to display the graph indicating the CT-FFR values and the graph indicating the Angio-FFR values in a superimposed manner. This process is realized, for example, as a result of the processing circuitry 55 invoking and executing the program corresponding to the controlling function 551 from the storage circuitry 54.

Subsequently, the obtaining function 552 judges whether or not a comparison display has been carried out with respect to all the target positions (step S107). When a comparison display has not been carried out with respect to all the target positions (step S107: No), the process returns to step S103 where the obtaining function 552 obtains Angio-FFR values corresponding to a new target position. On the contrary, when a comparison display has been carried out with respect to all the target positions (step S107: Yes), the medical image processing apparatus 5 ends the process. This process is realized, for example, as a result of the processing circuitry 55 invoking and executing the program corresponding to the obtaining function 552 from the storage circuitry 54.

Next, details of the processes performed by the medical image processing apparatus 5 will be explained.

### The CT-FFR Value Obtaining Process

As explained at step S101 in FIG. 3, in response to the CT-FFR value obtaining operation performed via the input interface 52, the obtaining function 552 is configured to obtain the CT-FFR values of the patient undergoing the treatment. For example, the obtaining function 552 is configured to obtain the CT-FFR values calculated with respect to the various positions along the extending direction of the coronary artery of the patient.

In this situation, the obtaining function 552 is configured to obtain information keeping the various positions in the coronary artery in correspondence with the CT-FFR values. In other words, the obtaining function 552 is configured to obtain the information with which it is possible to generate the graph indicating changes in the CT-FFR value with respect to the various positions in the coronary artery. In an example, the information may include a diagnosis result (e.g., a blood flow obstructed location diagnosed as requiring treatment) based on the CT-FFR values.

The CT-FFR value obtaining process at step S101 may be started in response to the obtaining instruction received from the user via the input interface 52 as described above or may be started automatically. In the latter situation, for example, the obtaining function 552 is configured to automatically obtain the CT-FFR values of the patient, when the condition is satisfied where patient information of the patient undergoing the treatment is input.

Further, together with the CT-FFR value being obtained, the obtaining function 552 is also able to obtain the CT image used for calculating the CT-FFR values. When the obtaining function 552 has obtained the CT image, the controlling function 551 is able to generate a display image from the obtained CT image and to cause the display 53 to display the generated display image. For example, the controlling function 551 is configured to generate, from the obtained CT image, the display image indicating the blood flow obstructed location diagnosed as requiring treatment and to further cause the display 53 to display the generated display image.

### The Target Position Determining Process

As explained at step S102 in FIG. 3, the determining function 553 is configured to determine the target position on the basis of the ΔFFR values of the obtained CT-FFR values. More specifically, the determining function 553 determines, as the target position for the position alignment, the blood flow obstructed location which has a large ΔFFR value and was diagnosed as requiring treatment. In this situation, the determining function 553 may obtain the blood flow obstructed location diagnosed as requiring treatment from the CT-FFR values or may obtain the blood flow obstructed location from the diagnosis result obtained at the same time as the CT-FFR values were obtained.

In other words, the determining function 553 is able to calculate the ΔFFR values by calculating the difference in each of the various positions, while using the CT-FFR values in the various positions in the coronary artery obtained by the obtaining function 552 and to further determine the target position (the blood flow obstructed location) on the basis of the calculated ΔFFR values. In that situation, the determining function 553 is configured to further specify a blood vessel position corresponding to the determined target position. For example, the determining function 553 is configured to specify a position in the coronary artery corresponding to a position between two CT-FFR values exhibiting large ΔFFR values.

Alternatively, the determining function 553 is configured to obtain the blood flow obstructed location in the coronary artery from the diagnosis result obtained together with the CT-FFR values and to determine the obtained blood flow obstructed location as the target position. In that situation, because the ΔFFR values and the positions in the coronary artery that correspond thereto have already been specified, the determining function 553 is configured to determine the target position on the basis of these pieces of information.

In this situation, the determining function 553 is configured to specify all the target positions in the coronary artery. For example, with respect to each of blood vessel branches of the blood vessel, the determining function 553 is configured to determine a target position. In other words, when there are a plurality of blood flow obstructed locations requiring treatment, the determining function 553 is configured to determine all the locations as target positions.

### The Angio-FFR Value Obtaining Process

As explained at step S103 in FIG. 3, the obtaining function 552 is configured to obtain the Angio-FFR values of the patient. More specifically, the obtaining function 552 is configured to obtain Angio-FFR values based on an angiography image acquired at a stage (pre-surgery) after the patient enters the treatment room but before the treatment is started or Angio-FFR values based on an angiography image acquire at a stage (post-surgery) after the treatment is performed but before the patient exits the treatment room. For example, the obtaining function 552 is configured to obtain information keeping the various positions in the coronary artery in correspondence with the Angio-FFR values. In other words, the obtaining function 552 is configured to obtain the information with which it is possible to generate the graph indicating changes in the Angio-FFR value with respect to the various positions in the coronary artery.

In this situation, as for the graph of the index values related to the blood flow based on the CT image and the graph of the index values related to the blood flow based on the angiography image, the obtaining function 552 is configured to obtain the graphs calculated in substantially the same cardiac phase as each other. In other words, the obtaining function 552 is configured to obtain the index values while ensuring that the temporal phase in which CT-FFR is calculated matches the temporal phase in which Angio-FFR is calculated. In other words, the obtaining function 552 is configured to obtain the index values while ensuring that the cardiac phase in which CT-FFR is calculated matches the cardiac phase in which Angio-FFR is calculated. For example, when CT-FFR uses the phase (RR 70%) at a point in time when 70% of an R-R interval has elapsed since the start of an R-wave, the obtaining function 552 is configured to transmit information to the blood flow information calculating apparatus 4 so as to calculate Angio-FFR in the RR 70% phase. Further, the obtaining function 552 is configured to obtain the Angio-FFR values calculated in the RR 70% phase.

In this situation, the sequential order for obtaining the CT-FFR values and the Angio-FFR values is not limited to the order illustrated in FIG. 3. It is also acceptable to obtain the CT-FFR values after obtaining the Angio-FFR values. In that situation, for example, the obtaining function 552 is configured to select the cardiac phase for calculating the CT-FFR values, so as to match the cardiac phase in which Angio-FFR was calculated. For example, when Angio-FFR was calculated in the RR 80% phase, the obtaining function 552 is configured to obtain CT-FFR values calculated in the RR 80% phase.

In this situation, when the CT image used for calculating the CT-FFR values does not include an image corresponding to the cardiac phase of interest (e.g., a CT image in the RR 80% phase), because it is not possible to obtain the CT-FFR values in the RR 80% phase, the obtaining function 552 is configured to designate the cardiac phase of Angio-FFR so as to match the cardiac phase of CT-FFR.

As explained above, the obtaining function 552 is configured to obtain the index values while ensuring that the temporal phase for calculating CT-FFR match the temporal phase for calculating Angio-FFR. In this situation, similarly to the process of obtaining the CT-FFR values, the obtaining function 552 is also able to obtain the angiography image used for calculating the Angio-FFR values, together with the Angio-FFR values being obtained. When the obtaining function 552 has obtained the angiography image, the controlling function 551 is able to generate a display image from the obtained angiography image and to cause the display 53 to display the generated display image. For example, the controlling function 551 is configured to generate, from the obtained angiography image, the display image indicating a location corresponding to the blood flow obstructed location diagnosed as requiring treatment and to further cause the display 53 to display the generated display image.

### The Position Alignment Process

As explained at steps S104 and S105 in FIG. 3, the position alignment function 554 is configured to specify the feature point in the vicinity of the target position determined by the determining function 553 and to perform the position alignment by using the specified feature point. More specifically, by performing the position alignment at the feature point in the vicinity of the target position, the position alignment function 554 is configured to perform the position alignment between the graph of CT-FFR and the graph of Angio-FFR. In this situation, as explained above, the obtaining function 552 is configured to obtain the graphs calculated in substantially the same cardiac phase as each other. In other words, the position alignment function 554 is configured to perform the position alignment between the graphs calculated in substantially the same cardiac phase as each other.

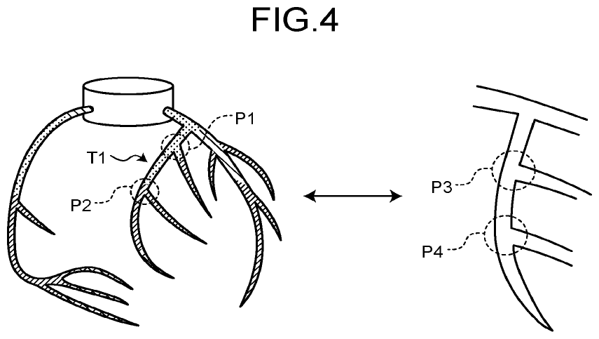

For example, in the coronary artery rendered in the CT image used for calculating the CT-FFR values and in the coronary artery rendered in the angiography image used for calculating the Angio-FFR values, the position alignment function 554 is configured to specify feature points each in the vicinity of the target position, and to further perform the position alignment so as to align the specified feature points with each other. FIG. 4 is a drawing for explaining an example of the position alignment process according to the first embodiment. In FIG. 4, the CT image used for calculating the CT-FFR values is illustrated on the left side, whereas the angiography image used for calculating the Angio-FFR values is illustrated on the right side.

For example, as illustrated on the left side of FIG. 4, the position alignment function 554 is configured to specify, in the CT image used for calculating the CT-FFR values, a blood vessel branch part P1 and a blood vessel branch part P2 as feature points in the vicinity of a target position T1 determined by the determining function 553. Further, as illustrated on the right side of FIG. 4, the position alignment function 554 is configured to specify, in the angiography image used for calculating the Angio-FFR values, a blood vessel branch part P3 corresponding to the blood vessel branch part P1 and a blood vessel branch part P4 corresponding to the blood vessel branch part P2.

After that, the position alignment function 554 is configured to perform the position alignment between the determined feature points. For example, the position alignment function 554 is configured to perform the position alignment by aligning the blood vessel branch part P1 with the blood vessel branch part P3 and aligning the blood vessel branch part P2 with the blood vessel branch part P4. In other words, the position alignment function 554 is configured to perform the process of superimposing the graphs together, by regarding the CT-FFR value in the blood vessel branch part P1 and the Angio-FFR value in the blood vessel branch part P3 as values in the same position and regarding the CT-FFR value in the blood vessel branch part P2 and the Angio-FFR value in the blood vessel branch part P4 as values in the same position.

In this situation, when performing the position alignment between the feature points (e.g., the blood vessel branch parts), the position alignment function 554 may perform an image deforming process. For example, when performing the position alignment to align the blood vessel branch part P1 with the blood vessel branch part P3 and to align the blood vessel branch part P2 with the blood vessel branch part P4, the position alignment function 554 may be configured to align the blood vessel branch part in one of the images with the blood vessel branch part in the other image, by stretching or shrinking the coronary artery in the one of the images along the extending direction. When having performed such a deforming process, the position alignment function 554 is configured to move the positions of the values in the FFR graph corresponding to the image which had the stretching or the shrinkage, to the positions corresponding to the stretching or the shrinkage.

For example, when a post-treatment Angio-FFR graph is compared with a CT-FFR graph, a medical tool such as a stent may be in place in many post-treatment situations. In those situations, because the shape of the blood vessel may have changed, the deforming process described above is performed so as to perform the position alignment with an excellent level of precision.

In the above description, the example was explained in which the location (the blood flow obstructed location) exhibiting a large ΔFFR value is used as the target position; however, possible embodiments are not limited to this example. For instance, it is also acceptable to use, as the target position, a point specified by other statistical analyses (e.g., an inflection point in the FFR graph or a point at which the value starts decreasing in the graph), a location of interest other than FFR selected prior to the surgery, a location previously treated, or the like.

In those situations, the determining function 553 is configured to determine one of the abovementioned points as the target position. Alternatively, the target position may be determined as a result of the controlling function 551 displaying a GUI for having the target position selected and receiving a target position selecting operation from an operator.

Further, in the above description, the example was explained in which the blood vessel branch parts are used as the feature points on which the position alignment is performed; however, possible embodiments are not limited to this example. For instance, it is also acceptable to use a point at which the value starts decreasing in the FFR graph. In other words, the position alignment function 554 may be configured to perform the position alignment between the CT-FFR graph and the Angio-FFR graph, by performing the position alignment at the feature points in the vicinity of target positions in the graphs.

Further, as the feature points between which the position alignment is performed, it is also acceptable to use a medical tool that was placed in previous treatment or the like. Also, it is also acceptable to extract a plurality of types of feature points such as blood vessel branch parts, a point at which the value starts decreasing in the FFR graph, a medical tool placed in previous treatment, and the like so as to perform the position alignment by aligning each of the extracted plurality of types of feature points. In addition, the feature points may be determined as a result of the controlling function 551 displaying a GUI for having the feature points selected and receiving a feature point selecting operation from the operator.

Further, in the above description, the example was explained in which the position alignment is performed between the images; however, possible embodiments are not limited to this example. For instance, a coronary artery model may be used. In that situation, for example, the position alignment function 554 may be configured to perform the position alignment between a feature point in the CT image and a feature point in the angiography image, by extracting a feature point (a blood vessel branch part) from the coronary artery model, aligning the position of the feature point in the CT image with the feature point in the coronary artery model, and aligning the position of the feature point in the angiography image with the feature point in the coronary artery model.

As explained above, the position alignment function 554 is configured to perform a local position alignment, by performing the position alignment while using the feature points in the vicinity of the target position. In other words, when there are two or more target positions, the position alignment function 554 is configured to perform the abovementioned position alignment with respect to each of the target positions. With this configuration, the position alignment function 554 is able to perform the position alignment on the target positions with a high level of precision.

Further, with respect to each of the blood vessel branches, the position alignment function 554 is configured to perform the position alignment between the graph of the index values related to the blood flow based on the CT image and the graph of the index values related to the blood flow based on the angiography image. Further, the position alignment function 554 is also capable of performing the position alignment on the entire coronary artery or on a range larger than the range used for aligning the blood vessel branch parts.

### The Comparison Display Process

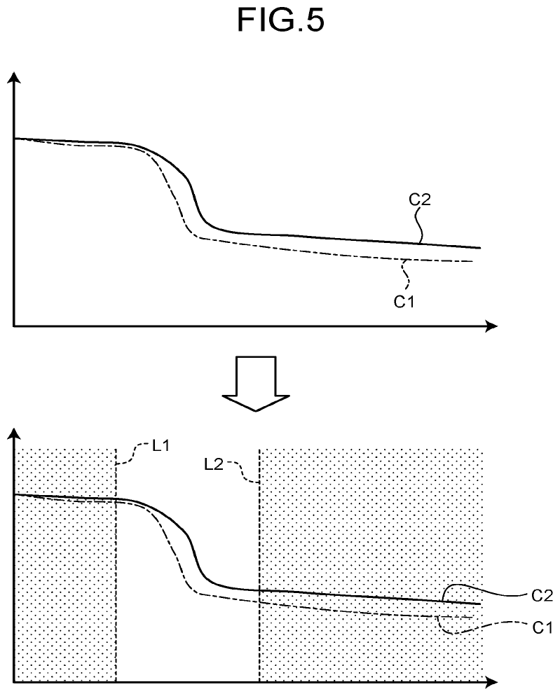

As explained at step S106 in FIG. 3, the controlling function 551 is configured to display, in the superimposed manner, the CT-FFR graph and the Angio-FFR graph on which the position alignment was performed by the position alignment function 554. FIG. 5 is a drawing illustrating an example of the comparison display according to the first embodiment. FIG. 5 presents graphs in which the horizontal axis expresses blood vessel positions, whereas the vertical axis expresses FFR values.

For example, as illustrated in the upper section of FIG. 5, the controlling function 551 is configured to present the comparison display of a CT-FFR graph C1 based on the CT image acquired pre-surgery and an Angio-FFR graph based on the angiography image acquired pre-surgery. In this situation, the controlling function 551 is configured to display the graph reflecting the position alignment performed by the position alignment function 554. In other words, the controlling function 551 is configured to display the graph in which FFR values between the feature points are arranged in the positions corresponding to the result of the position alignment.

For example, as illustrated in the bottom section of FIG. 5, the controlling function 551 is configured to display the graph in which the FFR values positioned between a straight line L1 indicating a position in the graph corresponding to the blood vessel branch part P1 (the blood vessel branch part P3) and a straight line L2 indicating a position in the graph corresponding to the blood vessel branch part P2 (the blood vessel branch part P4) are arranged in the positions corresponding to the result of the position alignment.

In this situation, the controlling function 551 is capable of further displaying information about a precision level of the position alignment, together with the graphs displayed in the superimposed manner. In other words, the controlling function 551 is capable of presenting a display in which an area having a higher level of precision for the position alignment is distinguishable from an area having a lower level of precision. For example, as illustrated in the bottom section of FIG. 5, the controlling function 551 is configured to indicate, in the graph, that the level of precision for the position alignment is lower by displaying, in gray, the areas outside the range (the area between the straight lines L1 and L2) on which the position alignment was performed.

### The Judging Process

As explained at step S107 in FIG. 3, the obtaining function 552 is configured to judge whether or not the comparison display was carried out with respect to all the target positions. More specifically, with respect to each of the target positions, the obtaining function 552 is configured to judge whether or not the comparison display was carried out for the CT-FFR graph with the pre-surgery Angio-FFR graph and with the post-surgery Angio-FFR graph. For example, the obtaining function 552 is configured to judge whether or not the comparison display was carried out, on the basis of identifiers distinguishing the pre-surgery result from the post-surgery result or elapsed time periods.

Further, with respect to each of the plurality of target positions, the obtaining function 552 is configured to judge whether or not the comparison display was carried out. In other words, with respect to each of the plurality of locations requiring treatment, the obtaining function 552 is configured to judge whether or not the comparison display was carried out. For example, the obtaining function 552 is configured to judge whether or not the comparison display was carried out with respect to each of all the target positions determined by the determining function 553.

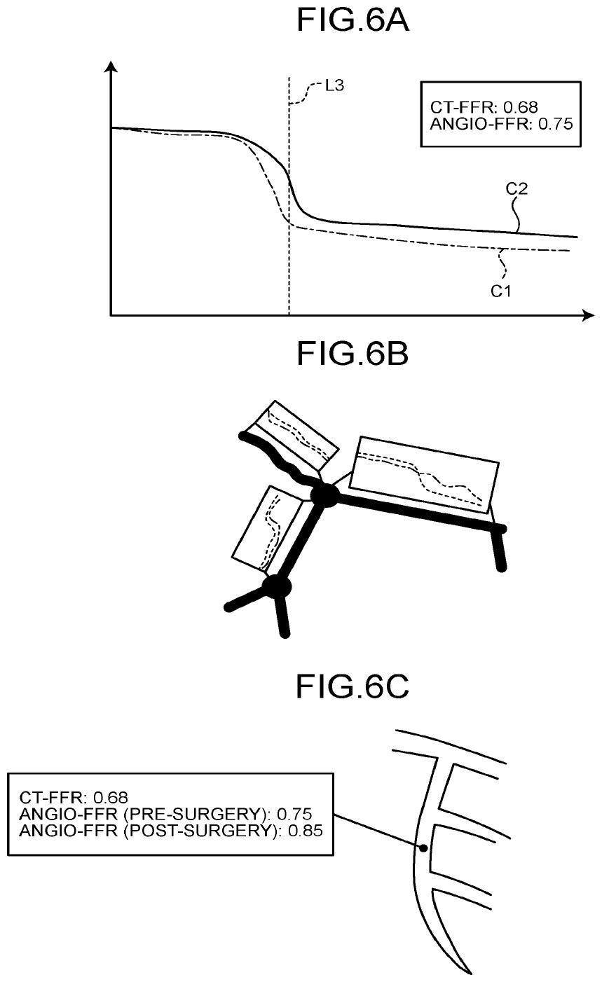

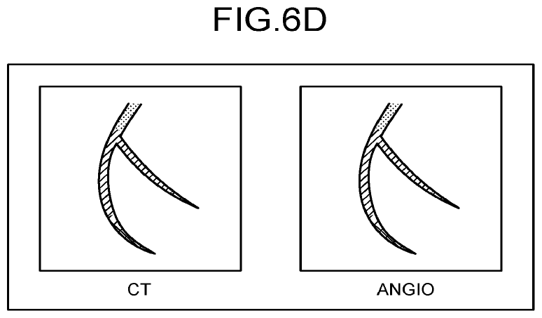

As explained above, the controlling function 551 is able to present the comparison display between the CT-FFR graph and the angio-FFR graph on which the local position alignment was performed. In addition to the comparison display of the graphs, the controlling function 551 is also capable of presenting other various types of display. Next, display control exercised by the controlling function 551 will be explained, with reference to FIGS. 6A to 6D. FIGS. 6A to 6D are drawings illustrating examples of the display control exercised by the controlling function 551 according to the first embodiment.

As illustrated in FIG. 6A, in addition to the comparison display between the CT-FFR graph and the Angio-FFR graph, the controlling function 551 is also capable of displaying the numerical values thereof. For example, as illustrated in FIG. 6A, the controlling function 551 is configured to display a GUI (a straight line L3) used for designating a position in which the numerical values are to be displayed. The operator designates the blood vessel position in which the numerical values are to be displayed, by moving the straight line L3 along the horizontal axis direction. The controlling function 551 is configured to display the numerical values representing the CT-FFR value and the Angio-FFR value in the position designated by the straight line L3.

Further, as illustrated in FIG. 6B, the controlling function 551 is also capable of displaying graphs so as to be kept in correspondence with blood vessel positions in a display image rendering the blood vessel. As explained above, the position alignment function 554 is configured to perform the position alignment with respect to each of the target positions. In other words, the controlling function 551 is capable of carrying out the comparison display between the CT-FFR graph and the Angio-FFR graph with respect to each of the target positions. Thus, as illustrated in FIG. 6B, the controlling function 551 is configured to present the comparison display between the CT-FFR graph and the Angio-FFR graph with respect to each of the target positions, so as to be kept in correspondence with the positions in the coronary artery.

Furthermore, as illustrated in FIG. 6C, besides displaying the graphs, the controlling function 551 is also capable of displaying, in numerical values, FFR values so as to be kept in correspondence with a position in an image of the coronary artery. For example, as illustrated in FIG. 6C, the controlling function 551 is configured to display a GUI (a point in the coronary artery) for designating a position in the coronary artery. The operator designates a blood vessel position in which the numerical values are to be displayed, by moving the point in the coronary artery along the extending direction. The controlling function 551 is configured to display, in the numerical values, a CT-FFR value and an Angio-FFR value in the position designated with the point in the coronary artery.

In addition, as illustrated in FIG. 6D, the controlling function 551 is configured to generate and display images of a blood vessel position corresponding to the target position, from the CT image and from the angiography image. In this situation, the controlling function 551 is configured to generate and display a blood vessel image from the CT image so as to have the same angle and the same size as the blood vessel rendered in the angiography image. Further, with respect to the blood vessel rendered in the images, the controlling function 551 is also capable of displaying a color map that is colored in accordance with the FFR values. In other words, the controlling function 551 is configured to display, side by side, a CT image indicating the blood vessel in colors corresponding to the CT-FFR values and an angiography image indicating the blood vessel in colors corresponding to the Angio-FFR values.

As explained above, the medical image processing apparatus 5 according to the present embodiment is configured to present the comparison display between the CT-FFR graph and the Angio-FFR graph. In this situation, in the comparison display between the CT-FFR graph and the Angio-FFR graph, the controlling function 551 is capable of displaying an alert corresponding to a comparison result, for the operator (a practitioner performing the treatment). For example, in the comparison display between the CT-FFR graph and the Angio-FFR graph, it may be difficult in some situations for the practitioner to notice that the results are different from each other. To cope with this situation, the controlling function 551 is configured to help viewers notice in the comparison display between the CT-FFR graph and the Angio-FFR graph, by displaying the alert in accordance with the comparison display between the plurality of graphs.

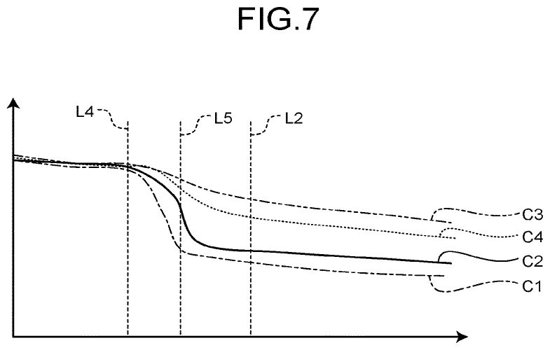

FIG. 7 is a drawing illustrating another example of the comparison display according to the first embodiment. For example, when the workflow has progressed up to the post-surgery stage so that the position alignment is performed with the post-surgery Angio-FFR, the controlling function 551 is capable, as illustrated in FIG. 7, of displaying, in a superimposed manner, a graph C1 of index values (CT-FFR) related to the blood flow based on the CT image acquired pre-surgery; a graph C3 of index values (Virtual CT-FFR) related to a post-surgery blood flow estimated by using the CT image acquired pre-surgery; a graph C2 of index values (Angio-FFR (pre-surgery)) related to the blood flow based on the angiography image acquired pre-surgery; and a graph C4 of index values (Angio-FFR (post-surgery)) related to the blood flow based on the angiography image acquired post-surgery.

In this situation, as illustrated in FIG. 7, at the time of sequentially displaying the four graphs in the order in which the graphs are generated, the controlling function 551 is configured to display an alert when a prescribed condition is satisfied in the comparison of the graphs. For example, the controlling function 551 is configured to display the alert, in accordance with a comparison result from a range being set on the basis of the target position, within the graph of the index values related to the blood flow. In an example, as illustrated in FIG. 7, the controlling function 551 is configured to display the alert, when a large difference is exhibited between the graphs, when FFR values are compared in the section between a straight line L4 and another straight line L5 indicating two inflection points in the CT-FFR graph C1.

For example, the controlling function 551 is configured to display the alert when there is a difference equal to or larger than a threshold value between the CT-FFR graph C1 and the Angio-FFR (pre-surgery) graph C2. With this arrangement, the controlling function 551 is able to help viewers notice that the result from CT-FFR is different from the result from Angio-FFR.

Further, the controlling function 551 is configured to display an alert when there is a difference equal to or larger than a threshold value between the virtual CT-FFR graph C3 and the Angio-FFR (post-surgery) graph C4. For example, the controlling function 551 is configured to display an alert when a value in the Angio-FFR (post-surgery) graph C4 does not reach a value in the virtual CT-FFR graph C3. With this arrangement, the controlling function 551 is able to help viewers notice that there is a possibility that the treatment may not be successful.

Further, in addition to the comparison of the FFR values between the straight line L4 and the straight line L5 indicating the two inflection points, the controlling function 551 is also capable of making comparisons in other positions and displaying an alert in accordance with the results thereof. For example, the controlling function 551 may be configured to compare the positions of inflection points on the horizontal axis between the graphs so as to display an alert when the difference is equal to or larger than a threshold value.

Further, the controlling function 551 may be configured to compare the values at a peripheral end between the graphs so as to display an alert when the difference is equal to or larger than a threshold value. In this situation, the scan controlling function 551 may compare the peripheral ends (the right ends) of the graphs with each other or may compare the peripheral ends (the positions at the straight line L2 in the drawing) of the area having a higher level of precision for the position alignment.

Further, when the post-treatment angiography image is acquired, the Angio-FFR values may be impacted by an injection of a drug, body movements, and/or the like, in some situations. To cope with those situations, when the compared results have a large difference, the controlling function 551 may suggest that Angio-FFR be re-calculated or may exercise control to automatically perform the re-calculation.

Further, in addition to displaying the alert, the controlling function 551 is also capable of displaying a numerical value indicating the difference in the FFR value between the compared graphs.

As explained above, according to the first embodiment, the obtaining function 552 is configured to obtain the graph of the index values related to the blood flow based on the CT image and the graph of the index values related to the blood flow based on the angiography image. On the basis of the shapes of the graphs of the index values related to the blood flow, the determining function 553 is configured to determine the target position for performing the position alignment between the graph of the index values related to the blood flow based on the CT image and the graph of the index values related to the blood flow based on the angiography image. On the basis of the target position, the position alignment function 554 is configured to perform the position alignment between the graph of the index values related to the blood flow based on the CT image and the graph of the index values related to the blood flow based on the angiography image. Consequently, the medical image processing apparatus 5 according to the first embodiment is able to perform the position alignment in the vicinity of the target position with an excellent level of precision and thus makes it possible to appropriately compare the index values related to the blood flow based on the CT image, with the index values related to the blood flow based on the angiography image.

Further, according to the first embodiment, the obtaining function 552 is configured to obtain the index value related to the blood flow in each of the various positions in the blood vessel rendered in the CT image and the index value related to the blood flow in each of the various positions in the blood vessel rendered in the angiography image. The determining function 553 is configured to determine the target position on the basis of the difference between the index values related to the blood flow with respect to each of the various positions in the blood vessel rendered in the CT image. Consequently, the medical image processing apparatus 5 according to the first embodiment is able to determine the target position on the basis of whether or not the blood flow is obstructed and to thus makes it possible to set the target position that is appropriate.

Further, according to the first embodiment, the determining function 553 is configured to determine the target position with respect to each of the blood vessel branches of the blood vessel. The position alignment function 554 is configured to perform, with respect to each of the blood vessel branches, the position alignment between the graph of the index values related to the blood flow based on the CT image and the graph of the index values related to the blood flow based on the angiography image. Consequently, the medical image processing apparatus 5 according to the first embodiment makes it possible to perform the position alignment with respect to each of the local positions.

Further, according to the first embodiment, as for the graph of the index values related to the blood flow based on the CT image and the graph of the index values related to the blood flow based on the angiography image, the obtaining function 552 is configured to obtain the graphs calculated in substantially the same cardiac phase as each other. The position alignment function 554 is configured to perform the position alignment between the graphs calculated in substantially the same cardiac phase as each other. Consequently, the medical image processing apparatus 5 according to the first embodiment enables the comparison having a high level of precision.

Further, according to the first embodiment, the determining function 553 is configured to specify the blood vessel position corresponding to the target position in the blood vessel. The position alignment function 554 is configured to perform the position alignment between the graph of the index values related to the blood flow based on the CT image and the graph of the index values related to the blood flow based on the angiography image, by performing the position alignment at the feature points in the vicinity of the blood vessel position. Consequently, the medical image processing apparatus 5 according to the first embodiment makes it possible to perform the local position alignment with an excellent level of precision.

Further, according to the first embodiment, the position alignment function 554 is configured to perform the position alignment between the graph of the index values related to the blood flow based on the CT image and the graph of the index values related to the blood flow based on the angiography image, by performing the position alignment at the feature points in the vicinity of the target position in the graphs. Consequently, the medical image processing apparatus 5 according to the first embodiment makes it possible to perform the position alignment using the graphs, with an excellent level of precision.

Further, according to the first embodiment, the controlling function 551 is configured to display, in the superimposed manner, the graph of the index values related to the blood flow based on the CT image and the graph of the index values related to the blood flow based on the angiography image on which the position alignment was performed by the position alignment function 554. Consequently, the medical image processing apparatus 5 according to the first embodiment makes it possible to present the comparison display having a high level of precision.

Further, according to the first embodiment, the controlling function 551 is configured to further display the information about the precision level of the position alignment, together with the graphs displayed in the superimposed manner. Consequently, the medical image processing apparatus 5 according to the first embodiment makes it possible to easily understand the locations in the graphs to be compared with each other.

Further, according to the first embodiment, the controlling function 551 is configured to display the graphs so as to be kept in correspondence with the blood vessel positions in the display image rendering the blood vessel. Consequently, the medical image processing apparatus 5 according to the first embodiment makes it possible to display the comparison between the images and the graphs.

Further, according to the first embodiment, the controlling function 551 is configured to display, in the superimposed manner, the graph of the index values related to the blood flow based on the CT image acquired pre-surgery; the graph of the index values related to the post-surgery blood flow estimated by using the CT image acquired pre-surgery; the graph of the index values related to the blood flow based on the angiography image acquired pre-surgery; and the graph of the index values related to the blood flow based on the angiography image acquired post-surgery. Consequently, the medical image processing apparatus 5 according to the first embodiment makes it possible to display the graphs obtained in the workflow as being put together in one chart.

Further, according to the first embodiment, the controlling function 551 is configured to display the alert in accordance with the comparison result between the plurality of graphs. Consequently, the medical image processing apparatus 5 according to the first embodiment makes it possible to help the practitioner notice.

Further, according to the first embodiment, the controlling function 551 is configured to display the alert in accordance with the comparison result in the area that is set, on the basis of the target position, in the graph of the index values related to the blood flow. Consequently, the medical image processing apparatus 5 according to the first embodiment makes it possible to help viewers notice in relation to important positions for the comparison.

### SECOND EMBODIMENT

In a second embodiment, a method for acquiring the angiography image used for calculating Angio-FFR will be explained. As explained in the first embodiment, in the workflow to which the present disclosure is applied, the CT image is acquired first, and it is checked to see whether or not a blood flow obstructed location is present, and if there is a blood flow obstructed location, the angiography image is acquired. Accordingly, at the time of acquiring the angiography image, the whereabout of the target position has already been recognized. Thus, in the second embodiment, the angiography image is acquired more efficiently, by acquiring the angiography image in accordance with the position of the blood flow obstructed location.

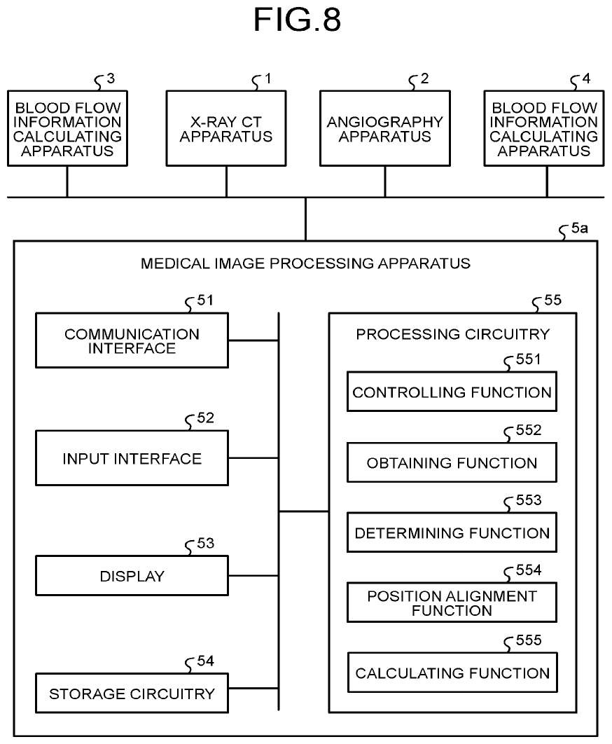

FIG. 8 is a diagram illustrating an exemplary configuration of a medical image processing apparatus 5a according to the second embodiment. The medical image processing apparatus 5a according to the second embodiment is different from the medical image processing apparatus 5 according to the first embodiment for newly including a calculating function 555 and for the control exercised by the obtaining function 552. In the following sections, these difference will primarily be explained. Some of the constituent elements that are the same each other will be referred to by using the same reference characters, and duplicate explanations thereof will be omitted.

On the basis of the target position in the blood vessel, the calculating function 555 according to the second embodiment is configured to calculate an acquisition direction of the angiography image with respect to the blood vessel. More specifically, the calculating function 555 is configured to calculate an X-ray emission direction (angle information of the arm) used for acquiring the angiography image taken of the blood flow obstructed location in the coronary artery determined on the basis of the CT-FFR values. Processes performed by the calculating function 555 will be explained in detail later.

The obtaining function 552 according to the second embodiment is configured to transmit, to the angiography apparatus 2, image taking condition setting information, so as to acquire the angiography image in the acquisition direction calculated by the calculating function 555. More specifically, the obtaining function 552 is configured to transmit the arm angle information calculated by the calculating function 555 to the angiography apparatus 2.

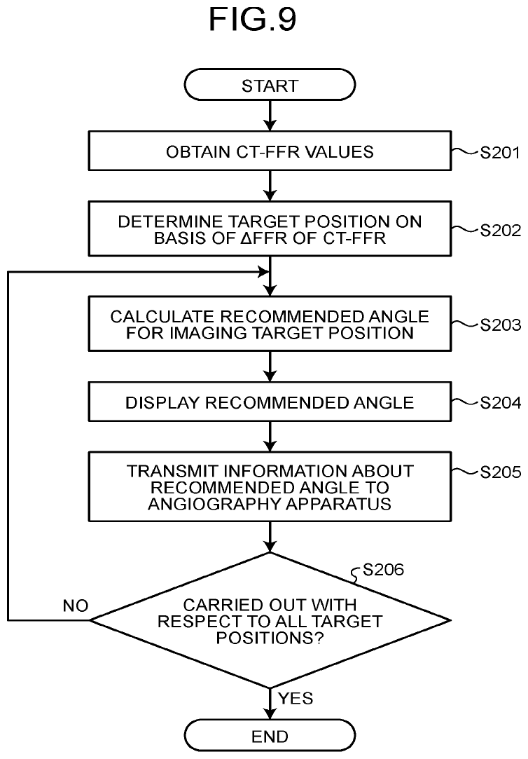

Next, a procedure in processes performed by the medical image processing apparatus 5a will be explained with reference to FIG. 9, and details of each of the processes will subsequently be explained. FIG. 9 is a flowchart illustrating the processing procedure of the processes performed by the processing functions included in the processing circuitry 55 of the medical image processing apparatus 5a according to the second embodiment. The flowchart illustrated in FIG. 9 is primarily applicable to a pre-surgery CAG scan performed after the patient determined to undergo treatment has entered a treatment room.

For example, as illustrated in FIG. 9, in the present embodiment, the obtaining function 552 obtains the CT-FFR values of the patient from the blood flow information calculating apparatus 3 (step S201). This process is realized, for example, as a result of the processing circuitry 55 invoking and executing the program corresponding to the obtaining function 552 from the storage circuitry 54.

Subsequently, the determining function 553 determines a target position on the basis of the ΔFFR values of the obtained CT-FFR values (step S202). This process is realized, for example, as a result of the processing circuitry 55 invoking and executing the program corresponding to the determining function 553 from the storage circuitry 54.

Subsequently, on the basis of the target position determined by the determining function 553, the calculating function 555 calculates a recommended angle for imaging the target position (step S203). This process is realized, for example, as a result of the processing circuitry 55 invoking and executing the program corresponding to the calculating function 555 from the storage circuitry 54.

After that, the controlling function 551 causes the calculated recommended angle to be displayed (step S204). This process is realized, for example, as a result of the processing circuitry 55 invoking and executing the program corresponding to the controlling function 551 from the storage circuitry 54.

Subsequently, the obtaining function 552 transmits information about the recommended angle to the angiography apparatus 2 (step S205). This process is realized, for example, as a result of the processing circuitry 55 invoking and executing the program corresponding to the obtaining function 552 from the storage circuitry 54.

After that, the obtaining function 552 judges whether or not a recommended angle has been calculated with respect to each of all the target positions (step S206). When recommended angles have not been calculated for all the target positions (step S206: No), the process returns to step S203, so that the calculating function 555 calculates a recommended angle with respect to a new target position. On the contrary, when a recommended angle has been calculated with respect to each of all the target positions (step S: Yes), the medical image processing apparatus 5 ends the process. This process is realized, for example, as a result of the processing circuitry 55 invoking and executing the program corresponding to the obtaining function 552 from the storage circuitry 54.

Next, details of the processes performed by the medical image processing apparatus 5a will be explained. Because the processes at steps S201, S202, and S206 in FIG. 9 are the same as the processes at steps S101, S102, and S107 in FIG. 3, the detailed explanations thereof will be omitted. The recommended angle calculating process

As explained at step S203 in FIG. 9, the calculating function 555 is configured to calculate the recommended angle for imaging the target position determined by the determining function 553. More specifically, the calculating function 555 is configured to calculate, as the recommended angle, a direction in which movements of the target position are relatively small (a direction in which moving amounts of the movements caused by pulsation are small) and which will cause no occurrence of foreshortening (a phenomenon where the length of the tool appears to be shorter) in the blood vessel at the target position.

For example, the calculating function 555 is configured to specify a positional relationship between the coronary artery and the target position on the basis of the target position determined in the CT image and the shape of the coronary artery. After that, on the basis of the specified positional relationship, the calculating function 555 is configured to calculate an angle with respect to the coronary artery (an angle with respect to the CT image (the volume data)) with which the target position satisfies the abovementioned conditions (a direction in which the movements are relatively small and which will cause no occurrence of foreshortening). After that, the calculating function 555 is configured to calculate the recommended angle by using the calculated angle with respect to the coronary artery.

In this situation, when the posture of the patient at the time of acquiring the angiography image is the same as the posture at the time of acquiring the CT image, the calculating function 555 is configured to determine, as the recommended angle, the same angle as the angle with respect to the coronary artery (the angle with respect to the CT image (the volume data)) within the image taking space of the angiography image.

Alternatively, when an angiography image of the patient has already been acquired in the treatment room, the calculating function 555 may be configured, for example, to extract the coronary artery from the already-acquired angiography image, on the basis of an extraction method based on the shape of the coronary artery. After that, the calculating function 555 is configured to calculate an angle within the image taking space of the angiography image with respect to the extracted coronary artery, the angle serving as the abovementioned angle calculated with respect to the coronary artery. The calculating function 555 is configured to determine the abovementioned angle within the angiography image taking space, as the recommended angle.

In the above example, the example was explained in which the determining function 553 is configured to determine the target position within the CT image; however, the operator may determine the target position. In that situation, the operator may further give a body site name of the target position, so that the calculating function 555 specifies an approximate position of the target position in the angiography image, on the basis of the given body site name.

### The Recommended Angle Display Process

As explained at step S204 in FIG. 9, the controlling function 551 is configured to cause the display 53 to display the recommended angle calculated by the calculating function 555. In this situation, the controlling function 551 is also capable of further having a GUI displayed for receiving approval on the angle from the operator.

### The Recommended Angle Transmitting Process

As explained at step S205 in FIG. 9, the obtaining function 552 is configured to establish a setting in the angiography apparatus 2 to acquire an angiography image at the recommended angle, by transmitting, to the angiography apparatus 2, the recommended angle calculated by the calculating function 555 as an image taking condition for the angiography apparatus 2.

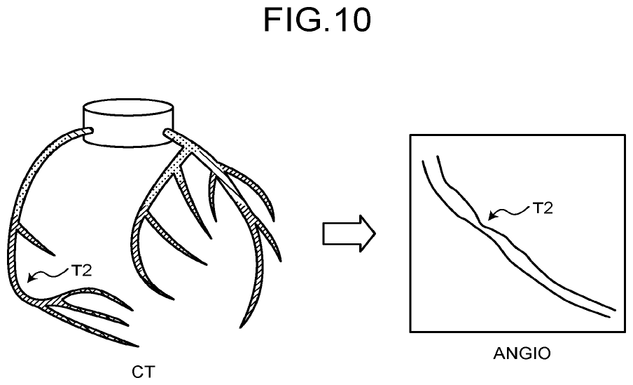

As a result of acquiring the angiography image with the abovementioned setting, it is possible to display the angiography image in which the target position can easily be seen at an initial display stage. FIG. 10 is a drawing for explaining an example of the image display according to the second embodiment. As illustrated in FIG. 10, when a target position T2 is determined in the CT image, it is possible to display the angiography image in which the target position T2 can easily be seen, due to the control based on the recommended angle described above. In other words, it is possible to display the angiography image in which the blood vessel at the target position T2 is indicated in the direction which causes no occurrence of foreshortening. Further, because the target position T2 is known, it is also possible to display an angiography image in which that position is enlarged.

Further, when the Angio-FFR value calculated by using the angiography image acquired in this manner is significantly different from the CT-FFR value, the obtaining function 552 is also capable of exercising control so that an angiography image is re-acquired or an Angio-FFR value is re-calculated. For example, the obtaining function 552 is configured to exercise control so that Angio-FFR is re-calculated after an angiography image is acquired at a different angle or changing the cardiac phase in which the imaging is performed.

Further, in the above description, the example was explained in which the angiography image is acquired in which the target position determined by the determining function 553 can easily be observed; however, possible embodiments are not limited to this example. For instance, with respect to a blood vessel of which an FFR value at a peripheral site is lower than a prescribed value, an angiography image may be acquired in which a location where a ΔFFR value exceeds a threshold value or a location set by the operator can easily be observed.

Further, when there are two or more target positions, the calculating function 555 is configured to calculate an angiography image acquisition direction with respect to each of the plurality of target positions. In that situation, the treatment will sequentially be carried out on the target positions. Thus, the medical image processing apparatus 5a is configured, with respect to each of the target positions, to calculate and display a recommended angle and to control the angiography image acquisition carried out by using the recommended angle. In this situation, while the treatment is sequentially carried out on the target positions, the controlling function 551 is capable of presenting each of the target positions on which the treatment has not yet been carried out and displaying the recommended angle for that position.

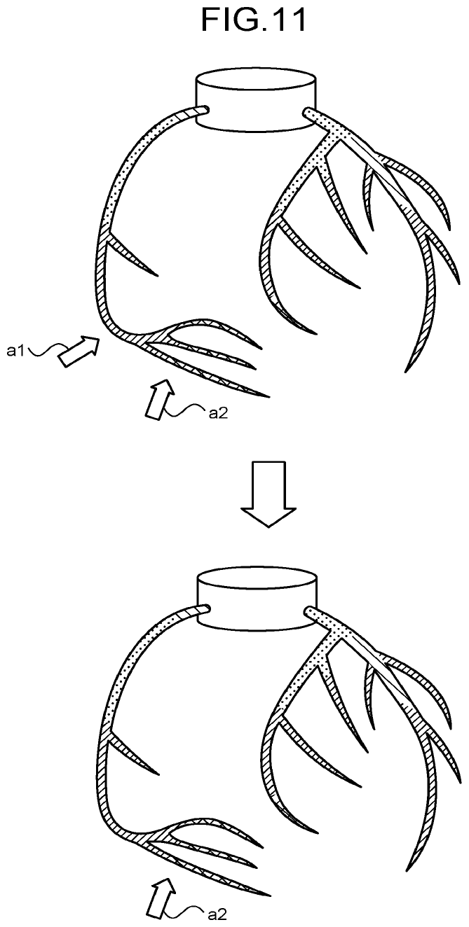

FIG. 11 is a drawing illustrating an example of display control according to the second embodiment. For example, as illustrated in the top section of FIG. 11, the controlling function 551 is configured to display an arrow a1 and an arrow a2 respectively indicating a plurality of target positions in the coronary artery. In this situation, for example, when the operator designates the position of the arrow a1, the controlling function 551 is configured to display a recommended angle for that position.

After that, when the treatment on the target position indicated by the arrow a1 is completed, the controlling function 551 is configured, as illustrated in the bottom section of FIG. 11, to make the arrow a1 disappear and to display only the arrow a2. Accordingly, the operator is able to clearly present the remaining location to be treated.

As explained above, according to the second embodiment, the determining function 553 is configured to determine the target position in the blood vessel rendered in the CT image, on the basis of the shape of the graph of the index values related to the blood flow based on the CT image. On the basis of the target position in the blood vessel, the calculating function 555 is configured to calculate the angiography image acquisition direction with respect to the blood vessel. Consequently, the medical image processing apparatus 5a according to the second embodiment makes it possible to easily acquire appropriate angiography images.

Further, according to the second embodiment, the determining function 553 is configured to determine the plurality of target positions in the blood vessel. The calculating function 555 is configured to calculate the angiography image acquisition direction with respect to each of the plurality of target positions. Consequently, even when there are a plurality of locations requiring treatment, the medical image processing apparatus 5a according to the second embodiment makes it possible to easily acquire an appropriate angiography image with respect to each of the locations.

### THIRD EMBODIMENT

In a third embodiment, a method for associating a CT-FFR calculation condition with an Angio-FFR calculation condition will be explained. More specifically, the method will be explained in which algorithms for CT-FFR and Angio-FFR are brought into collaboration, linked with each other, brought into coordination, and caused to exchange information with each other. The third embodiment is different from the first and the second embodiments for certain processes performed by the obtaining function 552. In the following sections, the differences will primarily be explained.

When calculating the index values related to the blood flow based on the CT image and the index values related to the blood flow based on the angiography image, the obtaining function 552 according to the third embodiment is configured to obtain graphs of the index values calculated under calculation conditions matching each other.

For example, the obtaining function 552 is configured to obtain a graph of the index values related to the blood flow based on the angiography image, the index values being calculated under a calculation condition matching that of the index values related to the blood flow based on the CT image. In an example, the obtaining function 552 is configured to exercise control so that an algorithm for the calculation of Angio-FFR is set so as to match an algorithm for the calculation of CT-FFR. For example, when the CT-FFR values are calculated by performing a fluid analysis using Navier-Stokes equations, the obtaining function 552 is configured to transmit setting information to the blood flow information calculating apparatus 4, so as to calculate Angio-FFR values through a fluid analysis using Navier-Stokes equations. In another example, when the CT-FFR values are calculated through an analysis using machine learning, the obtaining function 552 is configured to transmit setting information to the blood flow information calculating apparatus 4, so as to calculate Angio-FFR values through an analysis using machine learning.

Further, for example, the obtaining function 552 is configured to obtain information about a calculation range for the distal ends or a boundary condition used at the time of calculating CT-FFR and to further transmit the obtained information to the blood flow information calculating apparatus 4, and is thus able to obtain an Angio-FFR calculation result, by using a calculation range for the distal ends or a boundary condition matching that of CT-FFR.

Further, the obtaining function 552 is configured to obtain the graph of the index values related to the blood flow based on the CT image, the index values being calculated by using the calculation condition being set on the basis of the angiography image. For example, the obtaining function 552 is configured to transmit setting information to the blood flow information calculating apparatus 3, so that CT-FFR values are re-calculated while using a boundary condition matching that used for calculating Angio-FFR.

Further, from angiography images, it is possible to obtain a flow of a contrast agent by using a plurality of frames. Accordingly, the obtaining function 552 is configured to obtain a flow volume and a flow rate from the flow of the contrast agent and to further transmit setting information to the blood flow information calculating apparatus 3, so that CT-FFR values are re-calculated while using the obtained flow volume and flow rate as CT-FFR analysis conditions.

Further, from angiography images, it is possible to obtain the actual post-treatment shape of the blood vessel. Accordingly, the obtaining function 552 is configured to have CT-FFR calculated, by virtually deforming the blood vessel shape in the CT image into the post-treatment blood vessel shape in the angiography image. As a result, it is possible to obtain post-treatment angio-FFR values and the post-treatment CT-FFR values. Thus, by observing both types of the numerical values, it is possible to determine when to finish the treatment, with more confidence.

As explained above, according to the third embodiment, when calculating the index values related to the blood flow based on the CT image and the index values related to the blood flow based on the angiography image, the obtaining function 552 is configured to obtain the graphs of the index values calculated under the calculation conditions matching each other. Consequently, the medical image processing apparatus according to the third embodiment enables the comparison between the CT-FFR values and the Angio-FFR values obtained under the calculation conditions matching each other and thus makes it possible to compare the graphs more appropriately.

Further, according to the third embodiment, the obtaining function 552 is configured to obtain the graph of the index values related to the blood flow based on the angiography image, the index values being calculated under the calculation condition matching that of the index values related to the blood flow based on the CT image. Consequently, the medical image processing apparatus according to the third embodiment makes it possible to match the calculation condition of the Angio-FFR with the calculation condition of the CT-FFR.

Further, according to the third embodiment, the obtaining function 552 is configured to obtain the graph of the index values related to the blood flow based on the CT image, the index values being calculated by using the calculation condition being set on the basis of the angiography image. Consequently, the medical image processing apparatus according to the third embodiment makes it possible to match the calculation condition of CT-FFR with the calculation condition of Angio-FFR.

### OTHER EMBODIMENTS

In the above embodiments, the example was explained in which FFR is used as an index value related to the blood flow; however, possible embodiments are not limited to this example. For instance, it is possible to use, as an index value related to the blood flow, other index values indicating pressure, a pressure ratio, a flow volume, a flow ratio, fluid resistance, a fluid resistance ratio, iFR, or the like. Further, it is also acceptable to use an index value calculated by using any of the abovementioned index values in combination.

Further, in the above embodiments, the example was explained in which, for bringing CT-FFR into association with Angio-FFR, the calculation condition of CT-FFR is matched with the calculation condition of Angio-FFR. However, possible embodiments are not limited to this example. For instance, the obtaining function 552 may be configured to exercise control so that the shape of a three-dimensional coronary artery model used at the time of calculating Angio-FFR is corrected on the basis of the shape of a three-dimensional coronary artery model used at the time of calculating CT-FFR.

Further, for example, through machine learning that uses CT-FFR values, virtual CT-FFR values, and post-surgery Angio-FFR values as training data, it is also possible to construct a trained model configured to output a post-surgery angio-FFR value in response to an input of a CT-FFR value and a virtual CT-FFR value. By using this trained model, it is possible to simulate the post-surgery Angio-FFR value, on the basis of the CT-FFR value and the virtual CT-FFR values.

Further, it is also possible to structure the abovementioned processing circuitry 55 by combining together a plurality of independent processors, so that the processing functions are realized as a result of the processors executing the programs. Further, the processing functions of the processing circuitry 55 may be realized as distributed among or integrated into one or more pieces of processing circuitry as appropriate. Furthermore, the processing functions of the processing circuitry 55 may be realized by a combination of hardware such as circuitry and software. In addition, although the example was explained above in which the programs corresponding to the processing functions are stored in the single piece of storage circuitry (the storage circuitry 54), possible embodiments are not limited to this example. For instance, another configuration is also acceptable in which the programs corresponding to the processing functions are stored in a plurality pieces of storage circuitry in a distributed manner, so that the processing circuitry 55 reads and executes the programs from the pieces of storage circuitry.

Instead of realizing the display controlling unit, the obtaining unit, the determining unit, the position alignment unit, and the calculating unit of the present disclosure as the controlling function, the obtaining function, the determining function, the position alignment function, and the calculating function described in the embodiments, it is also acceptable to realize the functions by using hardware alone, software alone, or a combination of hardware and software.

The term “processor” used in the explanations of the above embodiments denotes, for example, a Central Processing Unit (CPU), a Graphics Processing Unit (GPU), or circuitry such as an Application Specific Integrated Circuit (ASIC) or programmable logic device (e.g., a Simple Programmable Logic Device (SPLD), a Complex Programmable Logic Device (CPLD), or a Field Programmable Gate Array (FPGA)). In this situation, instead of having the programs saved in the storage circuitry, it is also acceptable to directly incorporate the programs into the circuitry of one or more processors. In that situation, the one or more processors realize the functions by reading and executing the programs incorporated in the circuitry thereof. Further, the processors of any of the present embodiments do not each necessarily have to be structured as a single piece of circuitry. It is also acceptable to structure one processor by combining together a plurality of pieces of independent circuitry so as to realize the functions thereof.

In this situation, a medical image processing program executed by one or more processors is provided as being incorporated, in advance, in a Read Only Memory (ROM), storage circuitry, or the like. Alternatively, the medical image processing program may be provided as being recorded on a non-transitory computer-readable storage medium such as a Compact Disk Read-Only Memory (CD-ROM), a Flexible Disk (FD), a Compact Disk Recordable (CD-R), or a Digital Versatile Disk (DVD), in a file in a format that is installable or executable by those apparatuses. Further, the medical image processing program may be stored in a computer connected to a network such as the Internet so as to be provided or distributed as being downloaded via the network. For example, the medical image processing program may be structured with modules including the processing functions described above. In the actual hardware, as a result of a CPU reading and executing the medical image processing program from a storage medium such as a ROM, the modules are loaded into a main storage apparatus and generated in the main storage apparatus.

Further, in the embodiments and the modification examples described above, the constituent elements of apparatuses depicted in the drawings are based on functional concepts. Thus, it is not necessarily required to physically configure the constituent elements as indicated in the drawings. In other words, specific modes of distribution and integration of the apparatuses are not limited to those illustrated in the drawings. It is acceptable to functionally or physically distribute or integrate all or a part of the apparatuses in any arbitrary units, depending on various loads and the status of use. Further, all or an arbitrary part of the processing functions executed by the apparatuses may be realized by a CPU and a program analyzed and executed by the CPU or may be realized as hardware using wired logic.

In addition, with regard to the processes explained in the embodiments and the modification examples described above, it is acceptable to manually perform all or a part of the processes described as being performed automatically. Conversely, by using a publicly-known method, it is also acceptable to automatically perform all or a part of the processes described as being performed manually. Further, unless noted otherwise, it is acceptable to arbitrarily modify any of the processing procedures, the controlling procedures, specific names, and various information including various types of data and parameters that are presented in the above text and the drawings.

According to at least one aspect of the embodiments described above, it is possible to appropriately compare the index values related to the blood flow based on the CT image, with the index values related to the blood flow based on the angiography image.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A medical image processing apparatus comprising:
processing circuitry configured to
obtain a graph of an index value related to a blood flow based on a Computed Tomography (CT) image and a graph of an index value related to the blood flow based on an angiography image;
determine, on a basis of shapes of the graphs of the index values related to the blood flow, a target position for performing a position alignment between the graph of the index value related to the blood flow based on the CT image and the graph of the index value related to the blood flow based on the angiography image; and
perform, on a basis of the target position, the position alignment between the graph of the index value related to the blood flow based on the CT image and the graph of the index value related to the blood flow based on the angiography image.

2. The medical image processing apparatus according to claim 1, wherein the processing circuitry is configured to
obtain the index value related to the blood flow in each of various positions in a blood vessel rendered in the CT image and the index value related to the blood flow in each of the various positions in the blood vessel rendered in the angiography image, and
determine the target position on a basis of a difference between the index values related to the blood flow with respect to each of the various positions in the blood vessel rendered in the CT image.

3. The medical image processing apparatus according to claim 2, wherein the processing circuitry is configured to
determine the target position with respect to each of blood vessel branches of the blood vessel, and perform, with respect to each of the blood vessel branches, the position alignment between the graph of the index value related to the blood flow based on the CT image and the graph of the index value related to the blood flow based on the angiography image.

4. The medical image processing apparatus according to claim 1, wherein the processing circuitry is configured to obtain, as for the graph of the index value related to the blood flow based on the CT image and the graph of the index value related to the blood flow based on the angiography image, the graphs calculated in a substantially same cardiac phase as each other, and perform the position alignment between the graphs calculated in substantially the same cardiac phase as each other.

5. The medical image processing apparatus according to claim 2, wherein the processing circuitry is configured to specify a blood vessel position corresponding to the target position in the blood vessel, and perform the position alignment between the graph of the index value related to the blood flow based on the CT image and the graph of the index value related to the blood flow based on the angiography image, by performing the position alignment at a feature point in a vicinity of the blood vessel position.

6. The medical image processing apparatus according to claim 2, wherein the processing circuitry is configured to perform the position alignment between the graph of the index value related to the blood flow based on the CT image and the graph of the index value related to the blood flow based on the angiography image, by performing the position alignment at a feature point in a vicinity of the target position in the graphs.

7. The medical image processing apparatus according to claim 1, wherein the processing circuitry is configured to display, in a superimposed manner, the graph of the index value related to the blood flow based on the CT image and the graph of the index value related to the blood flow based on the angiography image on which the position alignment has been performed.

8. The medical image processing apparatus according to claim 7, wherein, together with the graphs displayed in the superimposed manner, the processing circuitry is configured to further display information about a precision level of the position alignment.

9. The medical image processing apparatus according to claim 7, wherein the processing circuitry is configured to display the graphs so as to be kept in correspondence with a position of a blood vessel in a display image rendering the blood vessel.

10. The medical image processing apparatus according to claim 7, wherein the processing circuitry is configured to display, in a superimposed manner, a graph of the index value related to the blood flow based on a CT image acquired pre-surgery; a graph of the index value related to a post-surgery blood flow estimated by using the CT image acquired pre-surgery; a graph of the index value related to the blood flow based on an angiography image acquired pre-surgery; and a graph of the index value related to the blood flow based on an angiography image acquired post-surgery.

11. The medical image processing apparatus according to claim 10, wherein the processing circuitry is configured to display an alert in accordance with a result of comparing two or more of the graphs.

12. The medical image processing apparatus according to claim 11, wherein the processing circuitry is configured to display an alert, in accordance with the result of the comparison made in a range being set in the graphs of the index values related to the blood flow on the basis of target position.

13. The medical image processing apparatus according to claim 1, wherein the processing circuitry is configured to
determine, on a basis of the shape of the graph of the index value related to the blood flow based on the CT image, the target position in a blood vessel rendered in the CT image, and
calculate, on a basis of the target position in the blood vessel, an acquisition direction of the angiography image with respect to the blood vessel.

14. The medical image processing apparatus according to claim 13, wherein the processing circuitry is configured to
determine, in the blood vessel, a plurality of target positions including the target position, and
calculate the acquisition direction of the angiography image with respect to each of the plurality of target positions.

15. The medical image processing apparatus according to claim 1, wherein, when calculating the index value related to the blood flow based on the CT image and the index value related to the blood flow based on the angiography image, the processing circuitry is configured to obtain graphs of the index values calculated under calculation conditions matching each other.

16. The medical image processing apparatus according to claim 15, wherein the processing circuitry is configured to obtain the graph of the index value related to the blood flow based on the angiography image, the index value being calculated under the calculation condition matching that of the index value related to the blood flow based on the CT image.

17. The medical image processing apparatus according to claim 15, wherein the processing circuitry is configured to obtain the graph of the index value related to the blood flow based on the CT image, the index value being calculated by using the calculation condition being set on a basis of the angiography image.

18. A medical image processing method comprising:
obtaining a graph of an index value related to a blood flow based on a Computed Tomography (CT) image and a graph of an index value related to the blood flow based on an angiography image;
determining, on a basis of shapes of the graphs of the index values related to the blood flow, a target position for performing a position alignment between the graph of the index value related to the blood flow based on the CT image and the graph of the index value related to the blood flow based on the angiography image; and
performing, on a basis of the target position, the position alignment between the graph of the index value related to the blood flow based on the CT image and the graph of the index value related to the blood flow based on the angiography image.

19. A storage medium storing therein, in a non-transitory manner, a program that causes a computer to execute:
obtaining a graph of an index value related to a blood flow based on a Computed Tomography (CT) image and a graph of an index value related to the blood flow based on an angiography image;
determining, on a basis of shapes of the graphs of the index values related to the blood flow, a target position for performing a position alignment between the graph of the index value related to the blood flow based on the CT image and the graph of the index value related to the blood flow based on the angiography image; and
performing, on a basis of the target position, the position alignment between the graph of the index value related to the blood flow based on the CT image and the graph of the index value related to the blood flow based on the angiography image.
